(19) United States
(12) Patent Application Publication
(10) Pub. No.: US 2013/0208862 A1
(43) Pub. Date: Aug. 15, 2013

(54) APPARATUS AND METHOD FOR DIGITAL RADIOGRAPHY

(71) Applicant: Robert A. Levine, Guilford, CT (US)

(72) Inventor: Robert A. Levine, Guilford, CT (US)

(21) Appl. No.: 13/768,888

(22) Filed: Feb. 15, 2013

Related U.S. Application Data
(60) Provisional application No. 61/599,187, filed on Feb. 15, 2012.

(51) Int. Cl. A61B 6/00

(52) U.S. Cl. CPC A61B 6/542
USPC 378/62

(57) ABSTRACT

A method and apparatus for digital radiography is provided. The method includes the steps of: a) providing an apparatus that includes an X-ray generator operable to generate and project X-rays, and a digital X-ray sensor operable to receive X-rays projected by the X-ray generator; b) determining whether the X-ray sensor is in an operable mode, or a non-operable mode; c) using the X-ray generator to project X-rays through a subject target area if the X-ray sensor is in the operable mode, or to prevent the generator from projecting X-rays through the subject target area if the X-ray sensor is in the non-operable mode; and d) using the X-ray sensor to receive the projected X-rays after they have transversed the subject target area if the X-ray sensor is in the operable mode.

This application claims the benefit of U.S. Patent Application Ser. No. 61/599,187, filed Feb. 15, 2012, which is hereby incorporated by reference into the present application in its entirety.

### BACKGROUND OF THE INVENTION

1. Technical Field

The present invention relates to methods and apparatuses for digital radiography in general, and to control techniques for digital radiography in particular.

2. Background Information

Digital radiography is an X-ray imaging technique in which digital X-ray sensors are used instead of traditional photographic film. Digital radiography may be preferred over traditional radiography because it eliminates the need for chemical processing of photographic film, it is more time efficient, and it enables digital transfer and enhancement of X-ray images. Digital radiography may be problematic, however, in that digital X-ray sensors are subject to inoperability; e.g., inoperability caused by hardware failure, battery failure, or absence of the digital X-ray sensor. Inoperability of a digital X-ray sensor is often not discovered until after digital radiography has been attempted. For example, in a common scenario, the digital radiography technician unsuccessfully attempts to image a patient, and discovers the inoperability of the digital X-ray sensor only after the patient has been irradiated with X-rays. Inoperability of a digital X-ray sensor is problematic, therefore, in that it can result in unnecessary irradiation of the patient with X-rays, which may be harmful to the patient in large doses. Inoperability of a digital X-ray sensor is also problematic because it causes lost practice and patient time, lost practice revenue, patient unhappiness and worry, and patient loss of confidence.

What is needed, therefore, is an improved apparatus and method for digital radiography, which apparatus and method prevents unnecessary irradiation of the patient with X-rays as result of digital X-ray sensor inoperability.

### SUMMARY OF THE INVENTION

According to an aspect of the present invention, an apparatus for digital radiography is provided that includes an X-ray generator operable to generate and project X-rays, and a digital X-ray sensor operable to receive X-rays projected by the X-ray generator. The X-ray generator and the X-ray sensor are operable to determine whether the apparatus is in an inoperable mode or an operable mode by communications between the X-ray sensor and the X-ray generator.

According to another aspect of the present invention, a method for digital radiography is provided. The method includes the steps of: a) providing an apparatus that includes an X-ray generator operable to generate and project X-rays, and a digital X-ray sensor operable to receive X-rays projected by the X-ray generator; b) determining whether the X-ray sensor is in an operable mode, or a non-operable mode; c) using the X-ray generator to project X-rays through a subject target area if the X-ray sensor is in the operable mode, or to prevent the generator from projecting X-rays through the subject target area if the X-ray sensor is in the non-operable mode; and d) using the X-ray sensor to receive the projected X-rays after they have transversed the subject target area if the X-ray sensor is in the operable mode.

The present apparatus and method, and advantages associated therewith, will become more readily apparent in view of the detailed description provided below, including the accompanying drawings.

### BRIEF DESCRIPTION OF THE DRAWINGS

FIG. 1 is a schematic view of the present apparatus.

FIG. 2 is a block diagram of steps that may be implemented using the present method.

### DESCRIPTION OF THE INVENTION

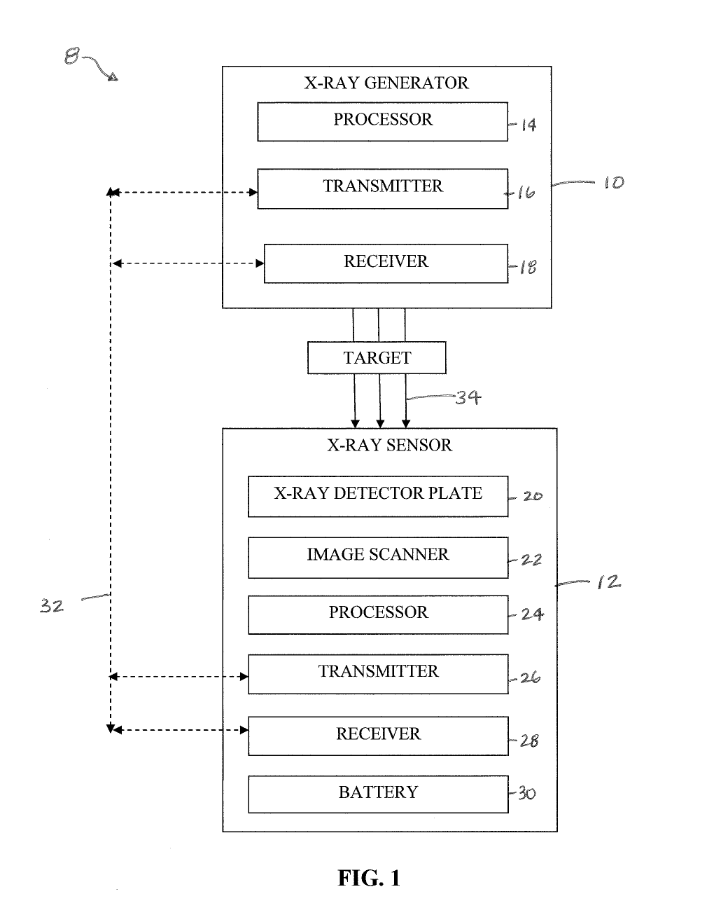

Referring to FIG. 1, the present invention provides an apparatus 8 and method for digital radiography. The apparatus 8 comprises an X-ray generator 10 and a digital X-ray sensor 12.

The X-ray generator 10 (an embodiment of which is shown schematically in FIG. 1) includes hardware (e.g., an X-ray tube, a collimator, etc.) that is operable to generate and project X-rays. The X-ray generator 10 also includes a processor 14, a transmitter 16 and a receiver 18. The processor 14 is in electronic communication with, and is operable to control, the hardware, the transmitter 16 and the receiver 18. The transmitter 16 and receiver 18 are operable to transmit and receive signals to and from the X-ray sensor 12, respectively, as discussed below.

The X-ray sensor 12 (an embodiment of which is shown schematically in FIG. 1) shown is operable to receive X-rays projected by the X-ray generator 10. The X-ray sensor 12 may be characterized as a flat panel detector or a high-density line-scan detector, which terms are known in the art. In the exemplary embodiment shown in FIG. 1, the X-ray sensor 12 includes an X-ray detector plate 20, an image scanner 22, a processor 24, a transmitter 26, a receiver 28, and a battery 30. The X-ray detector plate 20 contains a material (e.g., silicon), which generates photoelectrons in response to X-rays received from the X-ray generator 10. The image scanner 22 is operable to detect the photoelectrons generated by the X-ray detector plate 20, and create image data there from. The processor 24 is in electronic communication with, and is operable to control, the X-ray detector plate 20, the image scanner 22, the transmitter 26, the receiver 28, and the battery 30. The transmitter 26 and receiver 28 are operable to transmit and receive signals (e.g., image data) to and from the X-ray generator 10, respectively, as discussed below. The battery 30 provides the electricity required to power the X-ray detector plate 20, the image scanner, the processor 24, the transmitter 26, and the receiver 28.

The X-ray generator 10 and the X-ray sensor 12 are in communication with one another (which communication paths 32 are diagrammatically shown as dashed lines in FIG. 1). The X-ray generator transmitter 16 is operable to transmit communications to the X-ray sensor 12, and the X-ray sensor receiver 28 is operable to receive said communications. Similarly, the X-ray sensor transmitter 26 is operable to transmit communications to the X-ray generator 10, and the X-ray generator receiver 18 is operable to receive the communications. The communications between the transmitters 16, 26 and receivers 18, 28 of the X-ray generator 10 and the X-ray sensor 12 may be via wired or wireless connections. In embodiments in which the communications between the transmitters 16, 26 and receivers 18, 28 of the X-ray generator 10 and the X-ray sensor 12 are via wireless connections, the communications may be radio-frequency communications. The radio-frequency communications may be band-limited so as to prevent interference with other devices in close proximity to the apparatus 8. The apparatus 8 may alternatively or additionally be configured so that the radio-frequency communications are distance-limited (i.e., prevented from travelling more than a predetermined distance from the apparatus 8).

The X-ray generator 10 and the X-ray sensor 12 are operable to communicate with one another to confirm the operability of the X-ray sensor 12; i.e. if the apparatus is in an inoperable mode or an operable mode. The X-ray sensor 12 may be deemed to be in an operable mode if the battery 30 is providing sufficient electricity to power the X-ray detector plate 20, the image scanner 22, the processor 24, the transmitter 26, and the receiver 28. The X-ray sensor 12 may be deemed to be in an operable mode if the X-ray sensor processor 24 is internally in normal electronic communication and is in control of the X-ray detector plate 20, the image scanner 22, the transmitter 26, the receiver 28, and the battery 30. The X-ray sensor 12 may be deemed to be in an operable mode if the X-ray sensor 12 is within a particular distance from the X-ray generator 10. The X-ray sensor 12 may be deemed to be in an operable mode if the identity of the X-ray sensor 12 is known to the X-ray generator 10; e.g., if an identifier (e.g., an identity code) associated with the X-ray sensor 12 matches an identifier stored in the X-ray generator 10. The X-ray sensor (and therefore the apparatus 8) may be determined to be inoperable, conversely, if any of the above parameters are not true; e.g. if the X-ray sensor 12 is not internally in normal electronic communication, and/or if the X-ray sensor 12 is outside a particular distance from the X-ray generator 10, and/or if the identity of the X-ray sensor 12 is not known to the X-ray generator 10, etc. In addition, the apparatus 8 may be determined to be in an inoperable mode by the X-ray sensor 12 communicating to the X-ray generator 10 that the X-ray sensor 12 is located at a position relative to the X-ray generator 10 wherein the X-ray sensor 12 is unable to receive X-rays projected by the X-ray generator 10 in an amount sufficient transverse a subject target area and create a clinically usable image.

The X-ray generator transmitter 16 is operable to send, and the X-ray sensor receiver 28 is operable to receive, communications requesting that the X-ray sensor 12 confirm the operability of the X-ray sensor 12. Such communications are processed by the X-ray sensor processor 24, and the X-ray sensor processor 24 may execute protocols stored in memory to determine the operability of the X-ray sensor 12; e.g., protocols within the X-ray sensor 12 may determine (as indicated above) if internal communications within the X-ray are acceptable, if the X-ray sensor 12 is within a particular distance from the X-ray generator 10, etc. Communications indicating the operability of the X-ray sensor 12 are transmitted by the X-ray sensor transmitter 26, and are received by the X-ray generator receiver 18. If the communications indicate that the X-ray sensor 12 is inoperable, the X-ray generator 10 may be prevented (e.g., by a safety controller, or by protocols executed by the X-ray generator processor 14) from generating and projecting X-rays 34. The X-ray generator 10 may be prevented from generating and projecting X-rays 34 until the X-ray generator 10 receives communications from the X-ray sensor 12 indicating that the X-ray sensor 12 is operable. The digital radiography technician may be notified of the inoperability of the X-ray sensor 12 by visual or audible warning, which may for example be generated by a display or indicator on the X-ray generator 10. This feature prevents unnecessary exposure of patient to X-rays 34 as a result of X-ray sensor 12 inoperability. This feature can be performed quickly (e.g., in one second or less), and thus will not cause disruption to the use of the apparatus 8 when the X-ray sensor 12 is confirmed to be operable.

In some embodiments, the X-ray sensor 12 may be configured to continuously transmit a radio-frequency communication with an identifier (e.g., an ID code) when it is operable. The X-ray generator 10 may be configured to check to make sure that X-ray sensor 12 is generating the radio-frequency communication before it is used to generate and project X-rays. If the X-ray generator 10 does not detect the radio-frequency communication, the X-ray generator 10 will be prevented from generating and projecting X-rays 34, as discussed above.

In some embodiments, the X-ray sensor may be adapted to be mounted in a reader which can communicate with the X-ray sensor and also communicate with the X-ray generator, or other device, via wired or wireless communications.

Since an X-ray generator 10 may also be used with X-ray film as well as digital X-ray sensors 12, in some embodiments the X-ray generator 10 may include an override switch to change to a film mode in which case the communication between the X-ray sensor 12 and X-generator 10 is not required for generation of X-rays. In these embodiments, the apparatus 8 preferably includes a means for communicating with a cassette holding the X-ray film. The communications between the X-ray generator and the cassette may operate in a manner similar to that described between the X-ray generator 10 and the X-ray sensor 12.

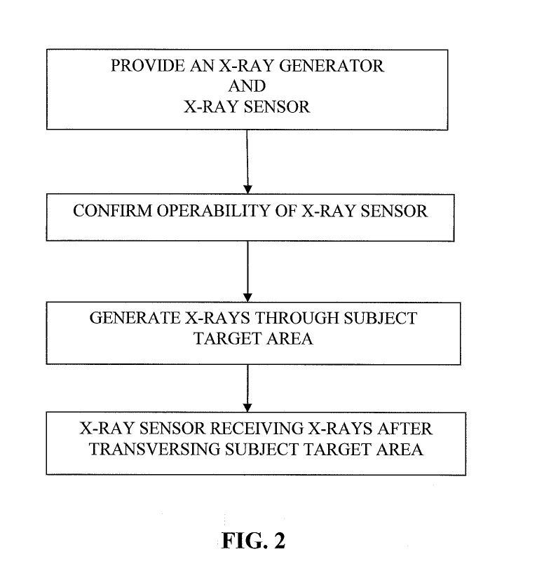

Now referring to FIGS. 1 and 2, the present invention also provides a method for digital radiography. The first step of the method involves providing an apparatus 8 that includes an X-ray generator 10 operable to generate and project X-rays 34, and a digital X-ray sensor 12 operable to receive X-rays 34 projected by the X-ray generator 10. For ease of description, the method will be described as being performed using the apparatus 8 described above. The method is not limited to use with this apparatus, however.

The second step involves confirming the operability of the X-ray sensor 12. This step may be performed as described above; e.g., the X-ray generator transmitter 16 may send, and the X-ray sensor receiver 28 may receive, communications requesting that the X-ray sensor 12 confirm the operability of the X-ray sensor 12, etc. In one example, the operability of the X-ray sensor 12 is confirmed, for example, by checking to make sure that the battery 30 is providing sufficient electricity to power the X-ray detector plate 20, the image scanner 22, the processor 24, the transmitter 26, and the receiver 28. If the battery 30 is providing insufficient electricity, this may be detected by the absence of a signal transmitted from the X-ray sensor 12 to the X-ray generator 10. Alternatively, the X-ray sensor 12 may continuously or periodically transmit a battery voltage measurement to the X-ray generator 10 for processing. This alternative approach may be preferred, as it allows the digital radiography technician to replace the battery 30 prior to failure. In another example, the operability of the X-ray sensor 12 may be confirmed by checking to make sure the X-ray sensor 12 is proximate to the X-ray generator 10; i.e., checking to make sure the X-ray sensor 12 is present and not, for example, outside of operable range (e.g., in another room). The X-ray generator 10 may check the proximity of the X-ray sensor 12 by detecting the presence of, and/or measuring the strength of, distance-limited radio-frequency communications transmitted by the X-ray sensor 12. The X-ray generator 10 may additionally or alternatively be configured to check the proximity of the X-ray sensor 12 to a desired position; e.g., a position such as the examination chair/table on which the patient is positioned during digital radiography. The X-ray sensor 12 and/or the X-ray generator 10 may also include directional antenna that are operable to ensure that the X-ray sensor 12 is not only proximate to the X-ray generator 10, but also correctly positioned relative thereto; e.g., the directional antenna may enable the X-ray generator 10 to confirm that the X-ray sensor 12 is within the path of the X-rays projected by the X-ray generator 10. In yet another example, the operability of the X-ray sensor 12 is confirmed by checking to make sure that a digital identity of the X-ray sensor 12 matches an identifier stored within the X-ray generator 10. This ensures that the X-ray generator 10 does not generate and project X-rays onto an unintended X-ray sensor 12 (e.g., an X-ray sensor 12 in use in an adjacent room).

The third step involves using the X-ray generator 10 to generate and project X-rays 34 through a subject target area. This step may be controlled by the X-ray generator processor 14, as described above.

The fourth step involves using the X-ray sensor 12 to receive the projected X-rays 34 after they have transversed the subject target area. This step may be controlled by the X-ray sensor processor 24, as described above.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed herein as the best mode contemplated for carrying out this invention.

What is claimed is:

1. An apparatus for digital radiography comprising:
an X-ray generator operable to generate and project X-rays; and
an X-ray sensor operable to receive X-rays projected by the X-ray generator;
wherein the X-ray generator and the X-ray sensor are operable to determine whether the apparatus is in an inoperable mode or an operable mode by communications between the X-ray sensor and the X-ray generator.

2. The apparatus of claim 1, wherein the X-ray generator and the X-ray sensor are operable to communicate with one another using radio-frequency communications.

3. The apparatus of claim 1, wherein the X-ray generator includes a safety controller operable to prevent the X-ray generator from generating and projecting X-rays if the apparatus is in the inoperable mode.

4. The apparatus of claim 1, wherein the X-ray generator includes a safety controller operable to prevent the X-ray generator from generating and projecting X-rays if the X-ray sensor and X-ray generator are not in communication with one another.

5. The apparatus of claim 1, wherein the X-ray sensor includes a battery, and the apparatus is determined to be the inoperable mode by the X-ray sensor communicating to the X-ray generator that the battery cannot produce adequate power to operate the X-ray sensor.

6. The apparatus of claim 1, wherein the apparatus is determined to be in the inoperable mode by the X-ray sensor communicating to the X-ray generator that the X-ray sensor is located beyond a threshold distance from the X-ray generator.

7. The apparatus of claim 1, wherein the apparatus is determined to be in the inoperable mode by the X-ray sensor communicating to the X-ray generator that the X-ray sensor is located at a position relative to the X-ray generator wherein the X-ray sensor is unable to receive X-rays projected by the X-ray generator in an amount sufficient transverse a subject target area and create a clinically usable image.

8. The apparatus of claim 1, wherein the apparatus is determined to be in the inoperable mode by the X-ray sensor communicating to the X-ray generator an identifier associated with the X-ray sensor that does not match an identifier stored within the X-ray generator.

9. The apparatus of claim 1, wherein the apparatus includes a safety indicator operable to indicate when the apparatus sensor is in the inoperable mode.

10. The apparatus of claim 1, wherein the X-ray generator is operable to generate X-rays when the communications between the X-ray generator and the X-ray sensor indicate that the apparatus is in the operable mode.

11. A method for digital radiography comprising the steps of:
providing an apparatus that includes an X-ray generator operable to generate and project X-rays, and a digital X-ray sensor operable to receive X-rays projected by the X-ray generator;
determining whether the X-ray sensor is in an operable mode, or a non-operable mode;
using the X-ray generator to project X-rays through a subject target area if the X-ray sensor is in the operable mode, or to prevent the generator from projecting X-rays through the subject target area if the X-ray sensor is in the non-operable mode; and
using the X-ray sensor to receive the projected X-rays after they have transversed the subject target area if the X-ray sensor is in the operable mode.

12. The method of claim 11, wherein the step of determining the mode of the X-ray sensor is performed by sending a radio-frequency communication from the X-ray generator to the X-ray sensor, and detecting a response communication generated by the X-ray sensor.

13. The method of claim 11, wherein the step of determining the mode of the X-ray sensor is performed by matching an identifier of the of X-ray sensor to an identifier stored within the X-ray generator.

14. The method of claim 11, wherein the step of determining the mode of the X-ray sensor is performed by determining if a battery associated with the X-ray sensor can produce adequate power to operate the X-ray sensor.

15. The method of claim 11, wherein the step of determining the mode of the X-ray sensor includes determining a distance between the X-ray sensor and the X-ray generator.
